(19) United States
(12) Patent Application Publication
(10) Pub. No.: US 2005/0031632 A1
(43) Pub. Date: Feb. 10, 2005

(54) Opposing blood group antibodies, foodstuffs and dietary supplements

(76) Inventor: Peter J. D'Adamo, New Canaan, CT (US)

Correspondence Address: ST. ONGE STEWARD JOHNSTON & REENS, LLC, 986 BEDFORD STREET, STAMFORD, CT, 06905-5619, US

(21) Appl. No.: 10/637,891

(22) Filed: Aug. 8, 2003

(51) Int. Cl. A61K 39/00

(52) U.S. Cl. 424/185.1

(57) ABSTRACT

A dietary supplement comprises substances selected so as not to trigger a blood type antibody response. A method of improving health of an individual comprises determining an individual's blood antigenicity and providing to said individual ingestible substances non-reactive to said blood antigenicity.

### FIELD OF THE INVENTION

The present invention relates to dietary supplements and methods of dietary supplementation. More specifically, the present invention relates to dietary supplement compositions and foods selected based on the user's blood type to minimize the presence of antigens which trigger an antibody reaction in the user's specific blood type.

### BACKGROUND OF THE INVENTION

Dietary supplements such as vitamins, minerals, herbs, probiotic compositions, amino acids and fatty acids, carbohydrates, glycoconjugates and other food products are historically used to provide nutritional supplementation to the user's normal diet. The effects of such dietary supplements vary greatly in effectiveness, but are nevertheless widely consumed for purposes such as improving functioning of the nervous, hormonal, immune, digestive, cardiovascular systems, and skeletal and joint systems. A number of dietary supplements are believed to improve health by improving effectiveness of the immune system. Such compositions are believed to support immune responses in fighting infection and disease.

I have observed certain relationships between human blood type, diet and illness and these observations were published in 1996 in my book entitled “Eat Right 4 Your Type.” This book, and my subsequent books disclose use of dietary supplements and the benefit of selecting foods and supplements that are of benefit for particular blood type. Blood type specific vitamins and dietary supplements have been sold with my name since at least 1999.

U.S. Pat. Nos. 6,291,533 and 6,503,529 to Fleischer claim to disclose dietary supplements and methods formulated for a human of specific antigen blood type. However, these supplements and methods do not address the problem of isoagglutinin production and the likelihood of an unnecessary immune response.

While dietary supplements have been designed to increase immune response and/or overall health, such dietary supplements do not address the possibility of a reaction of immune system based upon an individual's ABO blood type, nor do they consider the potential of unnecessary immune system reaction and the production of isoagglutinins by the immune system.

### SUMMARY OF THE INVENTION

It is an object of the invention to provide a dietary supplement and method which is selected to avoid the presence of blood group antigens which stimulate an antibody response. The supplement can be selected from the group consisting of vitamins, minerals, herbs, phytochemicals, botanicals, amino acids, and enzymes.

It is an object of the invention to provide a method of improving health of an individual by determining the blood type of the individual and providing to the individual a dietary supplement which does not contain a blood group antigen for such blood type.

Other objects, aspects and features of the present invention in addition to those mentioned above will be pointed out in or will be understood from the following detailed description.

### DETAILED DESCRIPTION

The human ABO blood group system is defined by the presence or absence of specific antigens in the blood. These unique carbohydrate or carbohydrate combinations found on the membrane of red blood cells (RBCs) define a person's blood type. The RBCs of a blood type O individual have on their surface the O-antigen, the sugar fucose, arranged in a long repeating chain. The RBCs of a blood type A individual have on their surface the base sugar fucose plus the carbohydrate N-acetyl galactosamine, the A antigen. The RBCs of a blood type B individual have on their surface the base sugar fucose plus the carbohydrate galactose (also called D-galactosamine), the B antigen. The RBCs of a blood type AB individual have on their surface the base sugar fucose plus both the A and B antigens, i.e. both N-acetyl galactosamine and galactose.

These RBC antigens are called isoantigens, a term for proteins or other substances that are present in only some members of a species and therefore able to stimulate antibody production in other members of the same species who lack the antigen. Humans who are exposed to foreign isoantigens, and antigens very similar thereto, produce antibodies that respond to the A and/or B antigens absent from their own RBCs. These are termed isoantibodies, and more specifically, isoagglutinins, the term for antibodies normally present in the sera of individuals that cause agglutination of the RBCs of another individual of the same species.

The immune system of a person of blood type A recognizes as foreign and will react to exposure to the B antigen, galactose, and produce anti-galactose antibody, called anti-B antibody or anti-B isoagglutinin. Likewise, the immune system if a person of blood type B will react to exposure to the A antigen, n-acetyl galactosamine, and will product anti-n-acetyl galactosamine antibody, called anti-A antibody or anti-A isoagglutinin. The immune system of a person of blood type AB will not react to exposure to either the A or B antigens, galactose or n-acetyl galactosamine, and produces no antibodies to them. In contrast, a person of blood type O recognizes both the A or B antigens, galactose or n-acetyl galactosamine, as foreign, and will produce both anti-A and anti-B antibodies/isoagglutinins.

The A and B antigens found in the molecules of human RBCs also exist in other biological entities, notably, bacterial cell walls, plants, and other foodstuffs. Bacteria are widespread in the environment, are present in intestinal flora, dust, food and other widely distributed agents, ensuring a constant exposure of individuals to A and B antigens and antigens that are extremely similar to each of these antigens. Many antigens or proteins in foods, such as lectins, have A-like or B-like characteristics and may likewise trigger an immune response and isoagglutinin production. This may explain why individuals who have not been otherwise exposed to antigen, for instance to incompatible blood via transfusion, will have a detectable isoagglutinin level in the blood stream. Isoagglutinin production may be a reaction to environmental provocations of antigens. Small amounts of A and B antigens may enter the body in food, bacteria, or by other means, and these substances initiate the development of isoagglutinins, e.g. the anti-A antibodies and/or anti-B antibodies. See Guyton, A. C., Textbook of Medical Physiology 8^{th} ed., W.B. Saunders Co., 1990.

Isoagglutinin production is generally seen after the first few months of life and continues throughout an individual's life, remaining fairly constant until late in adult life. See Liu, YJ et. al., The development of ABO isohemagglutinins in Tawanese. Hum. Hered. July/August 1996, 46(4):181-4. In the elderly, isoagglutinin production has been found to diminish and it is believed that this is due to the gradual reduction in efficiency of the immune defenses as the cells age. Recent studies measuring isoagglutinin levels suggest that the baseline isoagglutinin levels in children have risen overtime. See Godzisz, J., Synthesis of natural allohemagglutinins of the ABO blood system in healthy children aged 3 months to 3 years, Rev. Fr. Tranfus. Immunohematol., September 1979, 22(4): 399-412.

Studies have suggested that elevated issoagglutinin levels are linked with disease in humans, particularly those involving auto-immune dysfunction and/or excess inflammation, such as rheumatoid arthritis, endometriosis, chronic ear infection, Chrohn's disease of the intestines, asthma, eczema, and hives. See D'Adamo, P. Does ABO bias in innate immunity imply a different in T-cell reponse?, J. Naturopath. Med. 1991, 2:11-17.

It is possible that continual stimulation of the immune system by isoantigens and similar substances found in the environment and food unnecessarily consumes the resources of an individual's immune system and eventually reduces the immune systems ability to defend against real threats such as viruses, bacteria, and other harmful foreign antigens, thereby degrading overall health.

Additionally, unnecessary immune provocation and agglutinin production may lead to infertility in ABO incompatible couples. Studies have shown that agglutinin production in the mother may lead to “cervical hostility” to sperm from the ABO incompatible father. See Chakravartti, MR, et. al., ABO blood groups and fertility in an Indian population, J. Genet. Hum., June 1978, 26(2): 133-44; Cantuaria, AA., Blood group incompatibility and cervical hostility in relation to sterility, Obstet. Gynecol., February, 1978, 51(2):193-7; Mourant, A E, Blood Types and Diseases, Oxford Press, 1977.

I suggest that it is beneficial to overall health to avoid triggering an immune reaction where not required to respond to disease or other pathogens, or toxins, thus reducing production of antibodies and inflammation. The development of dietary substances and foodstuffs which are non-reactive to isoantigens could exert a resource-sparing effect on an individual's antibody defenses, prolonging immune competency over the course of his/her lifespan, modulating inflammation, improving fertility and enhancing host resistance.

Accordingly, one aspect of the invention comprises a dietary supplement for use by a human of a specific ABO blood type, said dietary supplement being selected and formulated to omit one or more of the A antigen and the B antigen and which would stimulate an antibody response in said human of specific ABO blood type. Providing a dietary supplement and/or foodstuffs that exclude substances which are foreign to and will provoke a immune response based upon an individual human's blood type and its resulting reactivity, will be a benefit to that individual's immune system, preventing unnecessary response and stress on the system, and will result in am overall improvement of health and well-being.

As used in this application, the term “dietary supplement” is used to identify the broad class of products to promote general health and wellbeing or promote functioning of a specific bodily organ, system, or other component thereof. Dietary supplements are ingested by mouth and contain any one or any combination of the following: vitamins, minerals, herbs, phytochemicals or other botanicals, amino acids, fatty acids, probiotics, glycoconjugates, substances such as enzymes and metabolites to supplement the diet by increasing the total dietary intake, concentrates, constituents or extracts.

Dietary supplements may take the form of a pill, tablet, capsule, softgel, gelcap, liquid, powder or other forms, such as a bar or drink, so long as it is not a conventional food or sole item of a meal or diet.

As used in this application, the term foodstuffs is used to identify conventional food products, such as those from conventional food groups such as grains (bread rice, cereal, pasta), protein (meats, poultry, fish, beans eggs and nuts), dairy (milk, cheese, yogurt), fruits and vegetables, and fats and oils, and other sundry dietary foodstuffs. Foodstuffs may naturally contain or may be enriched to contain the desired combination of vitamins, mineral, herbs or other botanicals, amino acids, carbohydrates, glycoconjugates, fatty acids, probiotic organisms, and forms of fiber. Herbs may be provided through tea.

Dietary supplements and foodstuffs are selected and/or formulated to avoid components that trigger an antibody response or other activation of the immune system of individuals of a particular blood type. The selected and/or formulated dietary supplements and foodstuffs are nonreactive to the immune system of individuals of a particular blood type thus preventing unnecessary immune reaction and isoagglutinin production.

In one embodiment of the present invention, supplements and foodstuffs formulated to support and improve the health of persons of blood type O and prevent production of anti-A and anti-B isoagglutinins exclude those substances, vitamins, minerals, herbals and other botanicals, amino acids, carbohydrates, glycoconjugates, fatty acids, that are similar to the A- and B-isoantigens and therefore will prompt an immune response.

Thus Vitamin A and vitamin E are excluded from dietary supplements formulated for blood type O individuals according to the present invention. To the extent Vitamin A is necessary for the individual's health, it may be obtained through normal diet, including consumption of yellow, orange, and dark leafy green vegetables. To the extent Vitamin E is necessary for the individual's health, it may be obtained through normal diet, so that Type O individuals may consume foods such as vegetable oils, liver, nuts, and leafy green vegetables.

Whole wheat products, domestic and shitake mushrooms, fermented olives, eggplant, potatoes, most melons, including cantaloupe and honeydew, blackberries, and coconut are excluded from foodstuffs for blood type O individuals according to the present invention.

Herbs excluded from dietary supplements and foodstuffs (e.g. herbal tea) for blood type O individuals according to the present invention are alfalfa, aloe, burdock, coltsfoot, corn silk, Echinacea, gentian, golden seal, red clover, rhubarb, Saint-John's wort, senna, shepherd's purse, strawberry leaf, and yellow dock.

Dietary supplements and foodstuffs to support and improve the health of persons of blood type O may contain vitamins, minerals, herbals and other botanicals, amino acids, carbohydrates, glycoconjugates, and fatty acids and that are known non-reactive with the B-isoantigen. Foodstuffs may naturally contain these components or be otherwise enriched.

Beneficial vitamins which are non-reactive to blood type O include vitamin B, specifically vitamin B-12 and folic acid, vitamin C, vitamin D and vitamin K. Foodstuffs according to the present inventions rich in vitamin B are meat, specifically liver, kidney, and muscle meats, eggs, fish, nuts, dark green leafy vegetables and fruit. Foodstuffs according to the present invention which are rich in vitamin K include liver, egg yolks, and green leafy vegetables such as kale, spinach and Swiss chard.

Beneficial herbs and phytochemicals known to be non-reactive to blood type O are licorice (Glycyrrhiza glabra), bladder wrack (Fucus vesiculosus) chickweed, dandelion, fengugreek, ginger, hops, linden, mulberry, parsley, peppermint, rose hips, sarsaparilla, and slippery elm. Additionally, dietary supplements beneficial and non-reactive to blood type O individuals may contain pancreatic enzymes.

Beneficial minerals known to be non-reactive to blood type O are calcium, iodine, manganese, and iron, provided by dietary supplement or appropriate foodstuffs according to the present invention include calcium and iodine, manganese and iron. According to the present invention, foodstuffs which are rich in calcium include sardines (unboned), canned salmon (unboned), broccoli, and collard greens. Foodstuffs which are rich in iodine include seafood, particularly salt water fish, kelp (seaweed), and iodized salt. Foodstuffs which are rich in manganese are whole grains, excluding wheat products, and legumes, excluding beans including copper, kidney, navy, tamarind, and lentils.

In another embodiment of the present invention, supplements and foodstuffs formulated to support and improve the health of persons of blood type A and prevent production of anti-B isoagglutinin are designed to exclude those substances, vitamins, minerals, herbals and other botanicals, amino acids, carbohydrates, glycoconjugates, fatty acids, that are similar to the B isoantigen and therefore will prompt an immune response.

Vitamin A, beta carotene, is excluded from dietary supplements formulated for blood type A individuals according to the present invention. To the extent Vitamin A is necessary for the individual's health, it may be obtained through normal diet, in foods such as eggs, yellow squash, carrots, spinach and broccoli.

Herbs excluded from dietary supplements and foodstuffs (e.g. herbal tea) formulated for blood type A individuals according to the present invention are catnip, cayenne, corn silk, red clover, rhubarb, and yellow dock.

Whole milk dairy products, white fish, such as sole and flounder, corn oil, cottonseed oil, peanut oil, safflower oil and sesame oil, copper beans, garbanzo beans, kidney beans, lima beans, navy beans, red beans, tamarind beans, peppers, fermented olives, domestic and sweet potatoes, yams, cabbage, tomatoes, and bananas are excluded from foodstuffs for blood type A individuals according to the present invention.

In another embodiment of the present invention, dietary supplements and foodstuffs to support and improve the health of persons of blood type A may contain vitamins, minerals, herbals and other botanicals, amino acids, carbohydrates, glycoconjugates, and fatty acids and that are not similar to the B-isoantigen and will prevent unnecessary anti-B isoagglutinin production. Foodstuffs may naturally contain these components or be otherwise enriched.

Beneficial vitamins which are non-reactive to blood type A include vitamin B, particularly vitamin B-12, vitamin C, and vitamin E. Foodstuffs according to the present inventions rich in vitamin B include whole grains (which contain niacin), soy sauce, miso, tempeh (all of which contain B-12), fish and eggs. Foodstuffs rich in vitamin C are berries, grapefruit, pineapple, cherries lemon and broccoli. Foodstuffs rich in vitamin E include vegetable oil, grains, peanuts, and leafy green vegetable.

Beneficial minerals known to be non-reactive to blood type A provided by dietary supplement or the appropriate foodstuffs according to the present include calcium, particularly calcium lactate and calcium citrate, iron, and zinc.

Foodstuffs containing calcium beneficial to individuals of blood type A according to this invention are yogurt, soy milk, eggs, goat milk, canned salmon with bones, sardines with bones, broccoli and spinach. Iron-rich foodstuffs according to the present invention include whole grains, beans, figs and blackstrip molasses.

Beneficial herbs and phytochemicals known to be non-reactive to blood type A and beneficial to the immune system are hawthorn, Echinacea, in particular purple coneflower (Echinacea purpurea), and huangki (Astragalus membranaceous). Other known beneficial herbs non-reactive to blood type A include chamomile, valerian root, quercetin, found in yellow onion, milk thistle, bromelain (found in pineapples), pro-biotic supplements (“good” bacteria high in bifidus factor), alfalfa, aloe, burdock, fenugreek, ginger, ginseng, green tea, rose hips, Saint-John's-Wort, slippery elm, and stone root, all of which may be ingested in the form of tea.

In another embodiment of the present invention, supplements and foodstuffs formulated to support and improve the health of persons of blood type B and prevent production of anti-A isoagglutinin are designed to exclude those substances, vitamins, minerals, herbals and other botanicals, amino acids, carbohydrates, glycoconjugates, fatty acids, that are similar to the A-isoantigen and therefore prompt an immune reaction.

Herbs which are excluded from dietary supplements and foodstuffs (e.g. herbal tea) formulated for blood type B individuals according to the present invention include aloe, coltsfoot, corn silk, fenugreek, gentian, goldenseal, hops, linden, mullein, red clover, rhubarb, senna, shepherd's purse, and skullcap.

Chicken, shellfish, peanut products, sesame products, sunflower products, corn products, lentils, garbanzo beans, pinto beans, black-eyed peas, rye products, olives tomatoes should be excluded from foodstuffs for blood type B individuals according to the present invention.

Dietary supplements and foodstuffs to support and improve the health of persons of blood type B may contain vitamins, minerals, herbals and other botanicals, amino acids, carbohydrates, glycoconjugates, and fatty acids and that are known non-reactive with the A-isoantigen. Foodstuffs may naturally contain these components or be otherwise enriched.

Beneficial vitamins nonreactive to blood type B include vitamins A, B, E, and C, which are preferable ingested by foodstuffs naturally containing such vitamins.

Beneficial minerals which are non-reactive to blood type B include magnesium. Foodstuffs according to the present inventions rich in magnesium include grains, legumes and green vegetables including collard greens, kale, mustard greens, parsley, parsnip, peppers, and brussel sprouts,

Beneficial herbs and phytochemicals known to be non-reactive to blood type B include licorice (Glycyrrhiza glabra), adaptogenic herbs such as, ginseng, specifically Siberian ginseng (Eleutherococcus senticosus) and Ginkgo biloba, licorice, ginger, parsley, peppermint, raspberry leaf, rose hips, sage, and lecithin which is found in soy and more effectively supplied by supplement. Additionally, dietary supplements beneficial and non-reactive to blood type O individuals may contain digestive enzymes, such as bromelain, which is found in foodstuffs, such as pineapple, or may be supplied by supplement.

Dietary supplements and foodstuffs to support and improve the health of persons of blood type AB may contain vitamins, minerals, herbals and other botanicals, amino acids, carbohydrates, glycoconjugates, and fatty acids and that are known non-reactive with the B-isoantigen. Foodstuffs may naturally contain these components or be otherwise enriched.

Herbs which are excluded from dietary supplements and foodstuff (e.g. herbal tea) formulated for blood type AB individuals according to the present invention are aloe, coltsfoot, corn silk, fenugreek, gentian, hops, linden, mullein, red clover, rhubarb, senna, shepherd's purse, and skullcap.

Chicken, white fish such as sole and flounder, kidney beans, lima beans, corn products, buckwheat, sesame products, sunflower products, mangoes, guava, and bananas are excluded from foodstuffs for blood type AB individuals according to the present invention.

Beneficial vitamins which are non-reactive to blood type AB include vitamin A, vitamin B-12, niacin, vitamin E, and vitamin C, which when provided by supplement is preferably derived from rose hips. Foodstuffs rich in vitamin C according to the present invention include berries, grapefruit, pineapple, cherries, lemon, and broccoli.

Beneficial herbs and phytochemicals known to be non-reactive to blood type AB include hawthorn (Crataegua oxyacantha), purple cornflower (Echinacea purpurea), huang-ki (Astragalus membranaceous), chamomile, valerian root, quercetin, milk thistle (Silybum marianum), and bromelain.

Beneficial minerals which are non-reactive to blood type AB provided by dietary supplement or foodstuffs according to the present invention include iron, zinc, and selenium. Foodstuffs according to the present inventions rich in zinc are meats, including dark-meat turkey, lamb, mutton, and rabbit, eggs and legumes.

It is to be appreciated that the foregoing is illustrative and not limiting of the invention, and that various changes and modifications to the preferred embodiments described above will be apparent to those skilled in the art. Such changes and modifications can be made without departing from the spirit and scope of the present invention, and it is therefore intended that such changes and modifications be covered by the following claims.

1. A dietary supplement for use by a human of a specific ABO blood type, said dietary supplement being selected and formulated to omit one or more of the A antigen and similar compounds and the B antigen and similar compounds and which would stimulate an antibody response in said human of specific ABO blood type.

2. The dietary supplement of claim 1, wherein the specific ABO blood type is blood type O.

3. The dietary supplement of claim 2, wherein the dietary supplement omits A antigens and B antigens.

4. The dietary supplement of claim 2, wherein the dietary supplement omits Vitamins A and E.

5. The dietary supplement of claim 1, wherein the specific ABO blood type is blood type A.

6. The dietary supplement of claim 5, wherein the dietary supplement omits B antigens.

7. The dietary supplement of claim 5, wherein the dietary supplement omits Vitamin A.

8. The dietary supplement of claim 1, wherein the specific ABO blood type is blood type B.

9. The dietary supplement of claim 8, wherein the dietary supplement omits A antigens.

10. The dietary supplement of claim 8, wherein the dietary supplement omits Vitamin E.

11. The dietary supplement of claim 8, wherein the dietary supplement omits aloe, coltsfoot, corn silk, fenugreek, gentian, goldenseal, hops, linden, mullein, red clover, rhubarb, senna, shepherd's purse, and skullcap.

12. A method of improving health of an individual comprising determining an individual's blood antigen type and providing to said individual ingestible substances selected to be nonreactive to said blood antigen type.

13. The method of claim 12, wherein said ingestible substances comprise foodstuffs.

14. The method of claim 12, wherein said ingestible substances comprise a dietary supplement.

15. The method of claim 14, wherein said dietary supplement comprises a pill, tablet, capsule, powder, or extract and providing said pill, tablet, capsule, powder or extract.

16. The method of claim 14, wherein said dietary supplement comprises one or more vitamins, minerals, herbs, phytochemicals, botanicals, amino acids, fatty acids, probiotics, glycoconjugates, and enzymes, and any combination thereof.

17. The method of claim 14, wherein the blood antigen type is blood type O.

18. The method of claim 17, wherein the dietary supplement omits A antigens and B antigens.

19. The method of claim 17, wherein the dietary supplement omits Vitamins A and E.

20. The method of claim 14, wherein the blood antigen type is blood type A.

21. The method of claim 20, wherein the dietary supplement omits B antigens.

22. The method of claim 20, wherein the dietary supplement omits Vitamin A.

23. The method of claim 14, wherein the blood antigen type is blood type B.

24. The method of claim 23 wherein the dietary supplement omits A antigens.

25. The method of claim 23 wherein the dietary supplement omits Vitamin E.

26. The method of claim 23 wherein the dietary supplement omits aloe, coltsfoot, corn silk, fenugreek, gentian, goldenseal, hops, linden, mullein, red clover, rhubarb, senna, shepherd's purse, and skullcap.
